(19) United States
(12) Patent Application Publication
(10) Pub. No.: US 2024/0378755 A1
(43) Pub. Date: Nov. 14, 2024

(54) SYSTEMS AND METHOD FOR SKIN COLOR DETERMINATION

(71) Applicant: FITSKIN INC., Toronto (CA)

(72) Inventors: Sergio RATTNER, Toronto (CA); Justinas VILIMAS, Vilnius (LT)

(21) Appl. No.: 18/696,662

(22) PCT Filed: Sep. 29, 2023
(86) PCT No.: PCT/CA2022/051443

§ 371 Date: Mar. 28, 2024

Related U.S. Application Data
(60) Provisional application No. 63/249,656, filed on Sep. 29, 2021.

(51) Int. Cl. G06T 7/90

(52) U.S. Cl. CPC G06T 7/90; G06T 2207/10024; G06T 2207/30088; G06T 2207/30196

(57) ABSTRACT

There is a system for user skin color determination of a user, the system comprising a skin analysis assembly, configured to obtain a user skin image of the user, comprising one or more user skin image pixels, perform color processing on the user skin image to determine a user skin color; and output the result of the color processing, the result comprising the user skin color.

### TECHNICAL FIELD

The present invention relates to measurement and analysis of skin color and in particular to systems and methods of measuring skin characteristics using skin analysis devices that attach to smartphones.

### BACKGROUND

Skin care product manufacturers create skin care products to assist users with maintaining healthy and beautiful skin. However, one of the biggest problems in the consumer skin care and cosmetics industries is determining an accurate and useful color of a consumer's skin, to be able to properly match and recommend cosmetics colors.

Various solutions exist that attempt to determine an accurate and useful skin color. However, limitations and failures of these solutions abound. For example existing solutions suffer from one or more of the following limitations:
(a) Inaccuracy in measurements. For example caused by technical limitations, unrealistic requirements of a user, changing environments (such as lighting) and the like.
(b) Cost. For example most solutions are stand-alone devices that do not leverage existing technology and are also high end components, such as spectrometers and spectrophotometers, which are very expensive and yet still suffer from various limitations described herein.
(c) Logistic challenges in deploying solutions. Specialized hardware is difficult to deploy, particularly when it is expensive. Hardware that is difficult to calibrate, maintain, or use is similarly difficult to deploy in a way that it will be used, and used accurately.

By way of example, one approach, that is considered desirable and accurate, is to use a spectrophotometer. Such a device does not use a camera and image analysis but rather uses wavelengths of light reflected to a sensor to determine a color of the subject (for example of a user's face). Such devices are expensive and are known to be effective for solid homogeneous colors, but not for non-homogeneous colors. Further, they're not effective the more translucent or brown or dark (ie high melanin) the skin is because the spectrophotometer overemphasizes capillaries (red/pink) or veins (blue/green).

There is accordingly a need in the art for an improved method and system capable of determining accurate skin colors using electronic devices such as smartphones.

### SUMMARY OF THE INVENTION

There is a system for user skin color determination of a user, the system comprising: a skin analysis assembly, configured to: obtain a user skin image of the user, comprising one or more user skin image pixels; perform color processing on the user skin image to arrive at a user skin color; and output a result of the color processing, the result comprising the user skin color.

The skin analysis assembly may comprise a skin analysis device attached to a mobile device.

The user skin image may be at a magnification of not less than 10×.

The performing color processing may further comprise: determining an average pixel condition color score for the user skin image pixels in the user skin image; and if the average pixel condition color score is below an image condition color threshold then: calculating a per-pixel condition color threshold; generating a condition color mask based on the per-pixel condition color threshold; and removing pixels, from the user skin image pixels, that exceed the per-pixel condition color threshold; and obtaining a user skin color from the user skin image pixels.

The performing color processing may further comprise: determining an average pixel redness score for the user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and obtaining a user skin color from the user skin image pixels.

The system determining may further comprise using a human perception mimicking algorithm developed using empirical A/B testing results.

The image redness threshold may be based on one or more of a processing power of the system, a desired speed and a desired accuracy of the calculated user skin color.

The calculating the per-pixel redness threshold may be by testing various per-pixel redness threshold values and choosing the per-pixel redness threshold value that minimizes the delta E from skin images that already had LAB values.

The system may be further configured to: apply a color transposition to the user skin color, based on a transposition between the system and an alternative skin color system, and wherein the result further comprises a transposed user skin color.

The system may be further configured to: provide a product recommendation, based on one or more of the user skin color and the transposed user skin color, and wherein the result further comprises a product recommendation.

The system may be further configured to: receive an empirical feedback, based on one or more of the product recommendation, the user skin color and the transposed user skin color.

The system may be further configured to: calibrate the skin analysis assembly, by taking one or more images of a calibration card, to obtain a calibration transposition that is applied to the user skin image.

There is also a method for user skin color determination of a user, the method comprising: obtaining, by a skin analysis assembly, a user skin image of the user, comprising one or more user skin image pixels; performing color processing on the user skin image to arrive at a user skin color; and outputting a result of the color processing, the result comprising the user skin color.

The skin analysis assembly may comprise a skin analysis device attached to a mobile device.

The user skin image may be at a magnification of not less than 10×.

The color processing may further comprise: determining an average pixel condition color score for the user skin image pixels in the user skin image; and if the average pixel condition color score is below an image condition color threshold then: calculating a per-pixel condition color threshold; generating a condition color mask based on the per-pixel condition color threshold; and removing pixels, from the user skin image pixels, that exceed the per-pixel condition color threshold; and obtaining a user skin color from the user skin image pixels.

The color processing may further comprise: determining an average pixel redness score for the user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and obtaining a user skin color from the user skin image pixels.

The determining may further comprise using a human perception mimicking algorithm developed using empirical A/B testing results.

The image redness threshold may be determined based on one or more of a processing power of the system, a desired speed and a desired accuracy of the calculated user skin color.

The calculating the per-pixel redness threshold may be by testing various per-pixel redness threshold values and choosing the per-pixel redness threshold value that minimizes the delta E from skin images that already had LAB values.

The method may further comprise: applying a color transposition to the user skin color, based on a transposition between the system and an alternative skin color system, and wherein the result further comprises a transposed user skin color.

The method may further comprise: providing a product recommendation, based on one or more of the user skin color and the transposed user skin color, and wherein the result further comprises a product recommendation.

The method may further comprise: receiving an empirical feedback, based on one or more of the product recommendation, the user skin color and the transposed user skin color.

The method may further comprise: calibrating the skin analysis assembly, by taking one or more images of a calibration card, to obtain a calibration transposition that is applied to the user skin image.

There is further a system for user skin color determination of a user, the system comprising: a skin analysis assembly, configured to: obtain a user skin image of the user, comprising one or more user skin image pixels; perform color processing on the user skin image to arrive at a user skin color, wherein the color processing further comprises: determining an average pixel redness score for the user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and calculating a user skin color from the user skin image pixels; and output a result of the color processing, the result comprising the user skin color.

The system may be further configured to: perform a color transposition to the user skin color, based on a transposition between the system and an alternative skin color system, and wherein the result further comprises a transposed user skin color.

The system may be further configured to: perform a product recommendation, based on one or more of the user skin color and the transposed user skin color, and wherein the result further comprises a product recommendation.

There is also a method for user skin color determination of a user, the method comprising: obtaining a user skin image of the user, comprising one or more user skin image pixels; performing color processing on the user skin image to determine a user skin color; and outputting the result of the color processing, the result comprising the user skin color.

The method may further comprise: determining an average pixel redness score user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and calculating a user skin color from the user skin image pixels.

The method may further comprise: performing a color transposition to the user skin color, based on a transposition between the system and an alternative skin color system, and wherein the result further comprises a transposed user skin color.

The method may further comprise: handling a product recommendation, based on one or more of the user skin color and the transposed user skin color, and wherein the result further comprises a product recommendation.

There is a system for user skin color determination of a user, the system comprising: a skin analysis assembly, configured to: obtain a user skin image of the user, comprising one or more user skin image pixels; perform color processing on the user skin image to determine a user skin color; and output the result of the color processing, the result comprising the user skin color.

The color processing may further comprise: determining an average pixel redness score for the user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and calculating a user skin color from the user skin image pixels.

The system may be further configured to: perform a color transposition to the user skin color, based on a transposition between the system and an alternative skin color system, and wherein the result further comprises a transposed user skin color.

The system may be further configured to: handle a product recommendation, based on one or more of the user skin color and the transposed user skin color, and wherein the result further comprises a product recommendation.

There is also a method for user skin color determination of a user, the method comprising: obtaining a user skin image of the user, comprising one or more user skin image pixels; performing color processing on the user skin image to determine a user skin color; and outputting the result of the color processing, the result comprising the user skin color.

The method may further comprise: determining an average pixel redness score user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and calculating a user skin color from the user skin image pixels.

The method may further comprise performing a color transposition to the user skin color, based on a transposition between the system and an alternative skin color system, and wherein the result further comprises a transposed user skin color.

The method may further comprise handling a product recommendation, based on one or more of the user skin color and the transposed user skin color, and wherein the result further comprises a product recommendation.

### BRIEF DESCRIPTION OF THE DRAWINGS

The invention is illustrated in the figures of the accompanying drawings which are meant to be exemplary and not limiting, in which like references are intended to refer to like or corresponding parts, and in which:

FIG. 1 illustrates aspects of a skin analysis system, according to an embodiment of the present invention;

FIGS. 2-4 are methods for skin color determination using a skin analysis system, according to aspects of the present invention; and

FIG. 5 is a sample result of a skin color reading using a skin analysis system as compared to conventional skin color reading.

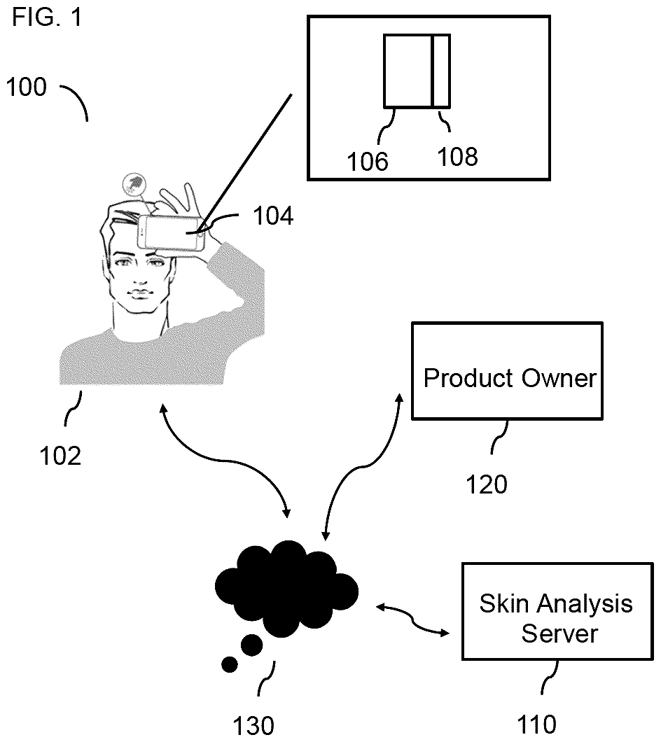

### DETAILED DESCRIPTION OF THE INVENTION

Broadly, system 100 comprises a skin analysis assembly 104 (which may comprise a skin analysis device 108 that attaches to a mobile device 106) that, when used by a user 102, performs one or more skin analysis actions such as capturing images of a user's face for color assessment, as described herein.

SAA 104 may be as described in PCT/CA2020/050216 or PCT/CA2017/050503, or may comprise another skin analysis system that is capable of taking images of a user's skin, the images having characteristics that are sufficient for the analysis described herein. It is to be understood that the skin analysis assembly 104 may have similar hardware components as described therein, and be able to interact and function similarly to as described in such references. SAA may have an application (app) thereon that user 102 can use to enable, control, or review methods described herein.

Notably, and as mentioned, system 100 requires the ability to obtain skin images that allow the processing described herein. In one embodiment, skin images may be taken using cross polarized light (for example to remove glare, or reflection of the light source from the skin image), with a 10 megapixel camera at a magnification of not less than 10×.

User skin images may be in one of several color formats, such as LAB or RGB. User skin images can be substantially of any quality, type, format or size/file size, provided the methods herein can be applied. For example, images may be compressed or not compressed, raw or processed, and in a variety of file formats.

SAS 110 may be a server that stores and processes skin characteristic measurements or samples, as described herein. SAS 110 may be any combination of web servers, applications servers, and database servers, as would be known to those of skill in the art. Each of such servers may comprise typical server components including processors, volatile and non-volatile memory storage devices and software instructions executable thereon. SAS 110 may communicate via app to perform the functionality described herein, including exchanging skin images, product recommendations, e-commerce capabilities, and the like. Of course app may perform these, alone or in combination with SAS 110, as well.

SAS 110 may include a database server that receives and stores all skin characteristic samples from all users into a user profile for each registered user 102 and guest user 102. These may be received from one or more SAA 104, though app may be configurable to store skin images locally only (though that may preclude some of the results information based on population and demographic comparisons).

SAS 110 may provide various analysis functionality as described herein (such as computing histograms of comparisons with a user's historical results or of comparisons with peers), and may provide various display functionality as described herein (such as providing websites that may present various analysis, provide links or functional links for other websites to access and display such results, recommendations and the like).

Product owners 120 may be entities that have skin care products, for example that can, or should, be adapted or selected based on a user's skin color. Product owners 120 may also have one or more product owner servers including web servers, applications servers, and database servers, as would be known to those of skill in the art. Each of such servers may comprise typical server components including processors, volatile and non-volatile memory storage devices and software executable thereon. Product owner 120 may be a point of communication for app (directly, or via SAS 110) for skin analysis measurement samples (such as those obtained via a user that was provided skin analysis device 108 by such product owner 120) and for storage and execution of product recommendation algorithms. For example, one or more generic product recommendation algorithms may be stored and owned by SAS 110 for each product recommendation type, and product owners may own and implement their own proprietary product recommendation algorithms (for example with product owner 120 receiving the required data to perform the product recommendation algorithm and returning the recommended product).

Product owners 120 may also offer e-commerce services directly, may suggest vendors such as Amazon™ (separately or with the recommended products) or may be agnostic about how a user may purchase a recommended product. Product owners 120 may also provide one or more e-commerce websites or screens (separate from or embedded in app) that facilitate business or commercial transactions involving the transfer of information over network 130 (such as the Internet). Types of e-commerce sites include but are not limited to: retail sites, auctions sites, and business-to-business sites. Exemplary e-commerce sites (that may be third parties, or not) that may facilitate the purchase of skin care products may include Amazon™, eBay™, and Overstock™. Of course product owners 120 may have their own e-commerce sites as part of their general websites, or SAS 110, that may provide system 100, may be such a vendor.

There may be other users 102 involved in system 102, such as beauty advisors or consultants, for example that work for product owners 120 or commerce sites or stores, that may assist a user 102 who is the subject of the image and whose skin color is being determined.

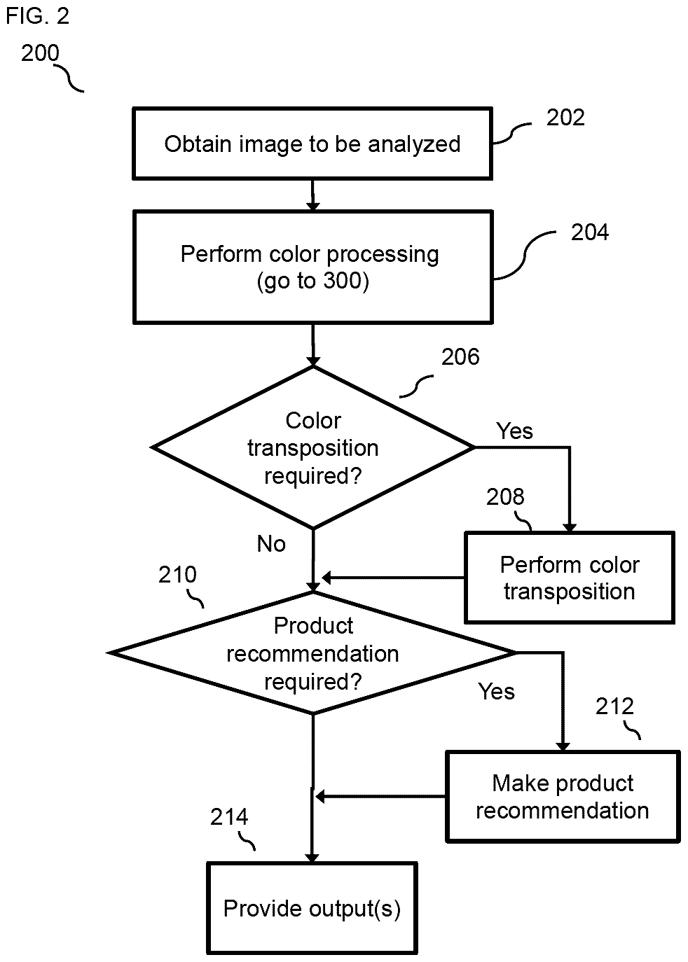

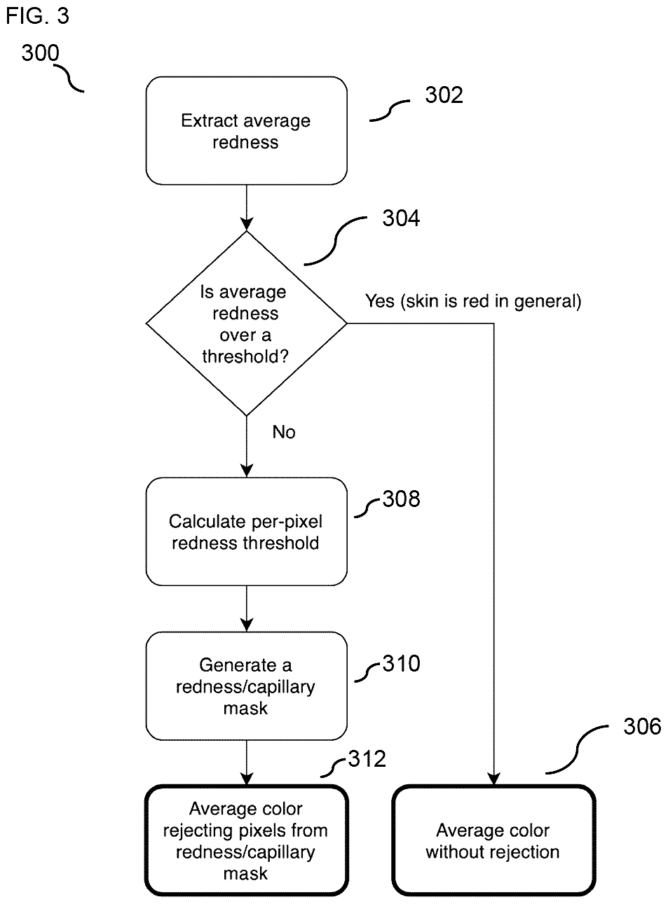

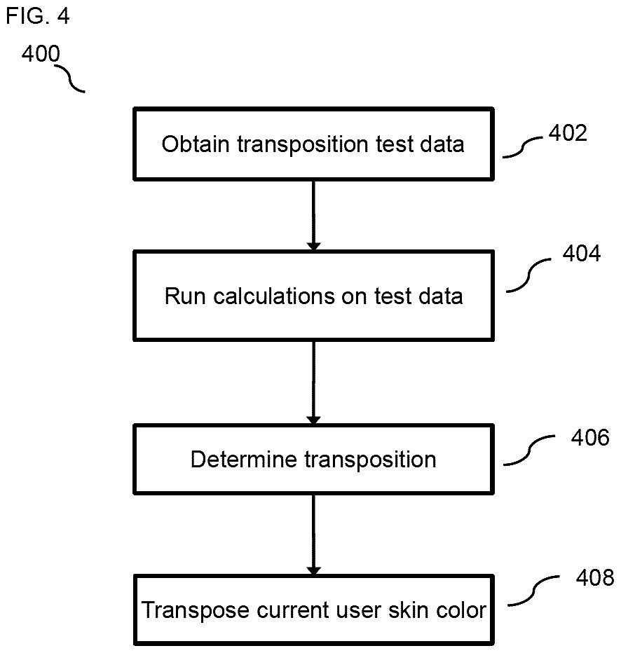

Turning to FIG. 2 there is a method 200 for skin color determination and processing. Method 200 begins at 202 where an image, as described herein, is obtained. This may be via skin analysis assembly 104 taking an image of user's 102 cheek.

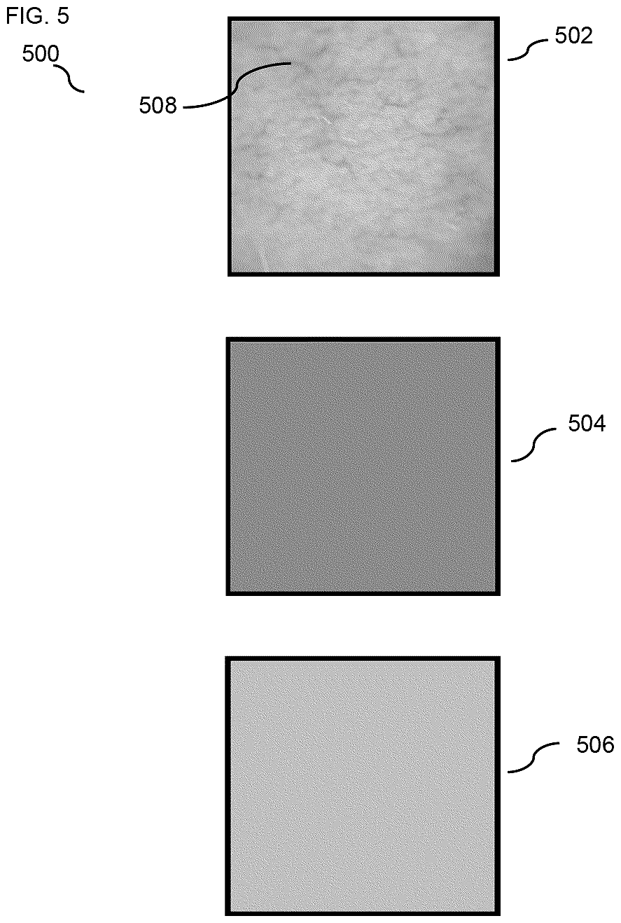

Having obtained a suitable image (such as image 502 in FIG. 5), method 200 continues at 204 to perform skin color processing. There may be one of several objectives for 204, but notably method 200 is seeking to determine an accurate skin color of the user 102 that is the subject of the skin image that was obtained at 202.

Skin color processing at 204 is further described, for example in method 300, beginning at 302 where an average redness score is determined for a user skin image. This may be done by taking the skin color score, largely the redness, for each pixel in the user skin image and calculating an average pixel redness.

The redness of each pixel, and then the average pixel redness, can be accomplished in various ways, with various impacts. A few approaches that were considered and tested include:
(a) Taking each pixel's redness from the value of the red channel (0-255) in RGB values (ie a score of 1-255, X, Y). The average is then summing the R value for each pixel and dividing by the number of pixels. In practice this method caused images with “redder” skin to have larger discrepancies from sample data results from another color measuring device.
(b) Research papers for directly calculating redness, or using “color distance” approaches (for example using (255, 0, 0) as a reference. Although these approaches showed some progress they still did not lead to as precise a result as was desired.
(c) Recognizing that human perception of redness may be different from algorithms, and that matching to human perception may provide a better ultimate color-matching experience (as human perception that there is a match may be more important than another determination), an algorithm was developed that mimicked human perceptions of redness was developed. Empirical evidence was collected that had subjects indicating which samples (which may be faces or may be rectangles of solid color) they perceived as being “redder”. In one such data capture, subjects were presented with several A/B tests, of colors or samples that appeared to be providing other algorithms problems (leading to less desirable outcomes). Problematic A/B options for algorithms tend to be where the “A” image had red channel values that were higher (ie mathematically “redder”) than the lower red channel value “B” options, but the “B” option appeared (and was empirically deemed to be) redder. Subjects chose A or B, for various examples of A/B combinations that appeared to be difficult for the algorithm, and the results of those selections were used to develop the human perception mimicking algorithm. The human perception mimicking algorithm could then be used to determine each pixel's redness, and then an average redness.

Method 300 then continues to 304 where the average pixel redness is compared to an image redness threshold and if the average pixel redness is above the threshold (meaning the user's skin may be reddish in general) then method 300 may not perform redness reduction and may proceed to 306 to determine a user skin color without rejecting or adjusting any pixels (ie the user skin color will be calculated using all pixels from the original user skin image). The threshold at 304 may be set at various scores depending on factors such as the processing power of system 100, the desired speed for a result, the need for accurate colors (for example some skin care products may not be particularly sensitive to slightly inaccurate color scores, or may not be affected by extra redness), and the like.

If the average pixel redness is below the image redness threshold, indicating that the user's skin is not generally reddish, then method 300 may proceed to 308. At 308 a pixel redness threshold (per-pixel redness threshold) is calculated—being the redness to which each pixel's redness score will be compared. The pixel redness threshold may be determined and set based on iterating through a large set of skin images that already had LAB values assigned to them and then testing using various threshold values for all those skin images and selecting the appropriate threshold, for example by minimizing the average deltaE (dE) between the existing LAB values and the LAB values obtained from the system, for the same image, and after using the particular redness threshold. Of course, the skin images with existing LAB values were subject to limitations of the capturing device, and further refinement may be possible with increased quality of the images, and capture device, used to establish the threshold.

The pixel redness threshold will be used to create a redness mask, which may also be known as a capillary mask. The redness mask may then be used to mask or block pixels that exceed the pixel redness threshold (or exceed it by enough), removing such pixels from the calculation of the skin image color or average skin image color.

Having created the redness mask, or otherwise having eliminated the pixels where the pixel redness exceeded the pixel redness threshold, method 300 continues to calculate the average score from the (remaining) pixels from the user skin image (ie the user skin color will be calculated based on the pixels that were not rejected as part of applying the redness mask). The averaging may be done using whatever color system is used—for example summing R, G and B scores for each unmasked pixel, and then dividing each of the R, G and B sums by the total number of unmasked pixels.

Method 300 ends either at 306 or 312 and then returns to 200. Although method 300 is shown in the figures as the primary method for calculating a skin image color, method 300 may simply be one processing technique that is applied to the user skin image. Others may exist, such as are described herein relating to blue coloring.

At this point, method 200 may substantially have a skin color for the user skin image. This may be adequate for the purposes being considered, and method 200 may bypass 206 and 208 to proceed directly to 214 to provide the output(s)—for example by providing a report of the user's skin color to a computing device or via email.

However, returning to method 200, at 206 method 200 determines whether color transposition is required. Color transposition involves, at a high level, determining what user skin color another skin analysis system would have determined for a particular user 102. For example, system 100 may determine a user skin color to be rgb (236, 188, 180) for a given skin image, while another system may have determined the same user's skin color to be rgb (209, 163, 164), regardless of whether it was determined by image or another technology (such as a spectrophotometer). In a specific example that was observed, as shown in FIG. 5, system 100 may determine a user skin color to be (239, 177, 163), as per image 506, based on image 502, where a separate technology (a spectrometer in particular) determines the user's skin color to be (175, 130, 110). Transposition may be desirable because a particular entity, such as product owner 120, may have a database of how given skin colors, determined by their skin color system, match to their products. However, they would not know how skin image colors, from system 100, may relate to those associations between their known skin color/product matches. Transposing system 100 user skin colors to product owner's 120 system may save considerable effort.

If transposing is desired at 206 method 200 continues to 400 where, at 402, transposition test data may be obtained. This may be done by obtaining sets of pairs of data where a given subject is assessed by system 100 and a particular other system to determine a color for each system. This may be repeated for enough subjects to ensure sufficient training data. This training data may be provided to an artificial intelligence or machine learning system, at 404, which may learn a transposition between the two systems at 406 (noting that system 100 may have multiple transpositions, such as one for each unique “alternative skin color assessment system”).

Having determined the transposition at 406, the transposition may be applied to the user skin color from method 200 so that a transposed user skin color may be obtained at 408. Both the user skin color and the transposed user skin color may be stored and used for various purposes, such as described herein.

Of course it is to be understood, and perhaps expected, that 402-406 may occur before method 200 commences, such that a transposition is known before a new user skin image is taken.

Method 400 ends and returns to method 200 at 210 where a query is made whether a product recommendation is required. If not, method 200 may end. If it is required then a product recommendation algorithm or lookup table may be consulted to determine what product to recommend for a particular user 102, based on one or more of their user skin color and/or transposed user skin color.

Of course methods described herein may further include feedback loops, including empirical feedback, to improve the systems and methods herein. Exemplary feedback may be:
(a) Enabling a user 102, such as a beauty advisor, to provide feedback (a score, an approval, etc) about how the recommended product appears to match the user 102 for whom it was recommended;
(b) Enabling a user 102, which maybe the subject, to provide feedback, such as a satisfaction score;
(c) Using any of the above feedback to refine a transposition that may have been used.

In method 300, redness caused by capillaries is taken into account and adjusted for so that the determined user skin color is not improperly affected by a system seeing additional red that is caused by capillaries but does not really represent the user's skin color. Similarly, veins may be observed by such systems, resulting in higher blue/green contributions to a user's skin color. A similar method to method 300 may be used to reduce the blue/green impact of veins—where a first determination is made of how “vein-like” a color is from RGB based values (similarly to for redness, as described), before a blue/green or “vein-like” threshold is determined so as to apply methods as described herein. In such case, an average pixel redness score may be replaced with an average pixel blueness score, an image redness threshold may be replaced with an image blueness threshold, a per-pixel redness threshold with a per-pixel blue threshold, a redness mask with a blueness mask, and so on. Although redness (capillaries) and blue/green (veins) are mainly contemplated, this approach may be used for other skin conditions, where the skin condition may distort algorithmic calculations of color, such that filtering out the effect of such skin condition may be desirable. As such, an average pixel redness score may be replaced with an average pixel condition color score, an image redness threshold may be replaced with an image condition color threshold, a per-pixel redness threshold with a per-pixel condition color threshold, a redness mask with a condition color mask, and so on.

Although not described in method 200, and not required for method 200, there may be a further calibration step, that may occur before method 200 begins. That may be to ensure that each skin analysis assembly 104 is adjusted to be the same as another—to ensure any system 100 that has the same SAA 104 will determine the same, or substantially similar, user skin colors. This may be accomplished by taking one or more images of a calibration card and determining a SAA 104 calibration transposition. This calibration transposition may be applied to a user skin image, for example before color processing is performed.

Returning briefly to FIG. 5, an image 502 (user skin image) is observed or taken, with various capillaries 508. Existing technology determines a skin color and outputs that as skin color image 504, with a value of (175, 130, 110). System 100 determines a skin color and outputs that as skin color image 504, with a value of (239, 177, 163). Although possibly better observed in color, images 502 and 506 are clearly more closely matched than 502 and 504. This is reinforced and validated with a deltaE calculation, where the deltaE between image 502 and image 504 is 18 while the deltaE between image 502 and image 506 is 3. As is known to those of skill in the art, it is generally accepted that a dE of 3 or less is imperceptible to the human eye.

The above-described embodiments of the present disclosure can be implemented in any of numerous ways. For example, the embodiments may be implemented using hardware, software or a combination thereof. When implemented in software, the software code can be executed on any suitable processor or collection of processors, whether provided in a single computer or distributed among multiple computers.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the concepts disclosed herein may be embodied as a non-transitory computer-readable medium (or multiple computer-readable media) (e.g., a computer memory, one or more floppy discs, compact discs, optical discs, magnetic tapes, flash memories, circuit configurations in Field Programmable Gate Arrays or other semiconductor devices, or other non-transitory, tangible computer storage medium) encoded with one or more programs that, when executed on one or more computers or other processors, perform methods that implement the various embodiments of the present disclosure discussed above. The computer-readable medium or media can be transportable, such that the program or programs stored thereon can be loaded onto one or more different computers or other processors to implement various aspects of the present disclosure as discussed above.

The terms “program”, “app” or “application” or “software” are used herein to refer to any type of computer code or set of computer-executable instructions that can be employed to program a computer or other processor to implement various aspects of the present disclosure as discussed above. Additionally, it should be appreciated that according to one aspect of this embodiment, one or more computer programs that when executed perform methods of the present disclosure need not reside on a single computer or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that conveys relationship between the fields. However, any suitable mechanism may be used to establish a relationship between information in fields of a data structure, including through the use of pointers, tags or other mechanisms that establish relationship between data elements.

Various features and aspects of the present disclosure may be used alone, in any combination of two or more, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Also, the concepts disclosed herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc. in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

Several (or different) elements discussed below, and/or claimed, are described as being “coupled”, “in communication with”, or “configured to be in communication with”. This terminology is intended to be non-limiting, and where appropriate, be interpreted to include without limitation, wired and wireless communication using any one or a plurality of a suitable protocols, as well as communication methods that are constantly maintained, are made on a periodic basis, and/or made or initiated on an as needed basis.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

This written description uses examples to disclose the invention and also to enable any person skilled in the art to make and use the invention. The patentable scope of the invention is defined by the claims, and may include other examples that occur to those skilled in the art. Such other examples are within the scope of the claims if they have structural elements that do not differ from the literal language of the claims, or if they include equivalent structural elements with insubstantial differences from the literal language of the claims.

It may be appreciated that the assemblies and modules described above may be connected with each other as required to perform desired functions and tasks within the scope of persons of skill in the art to make such combinations and permutations without having to describe each and every one in explicit terms. There is no particular assembly or component that may be superior to any of the equivalents available to the person skilled in the art. There is no particular mode of practicing the disclosed subject matter that is superior to others, so long as the functions may be performed. It is believed that all the crucial aspects of the disclosed subject matter have been provided in this document. It is understood that the scope of the present invention is limited to the scope provided by the independent claim(s), and it is also understood that the scope of the present invention is not limited to: (i) the dependent claims, (ii) the detailed description of the non-limiting embodiments, (iii) the summary, (iv) the abstract, and/or (v) the description provided outside of this document (that is, outside of the instant application as filed, as prosecuted, and/or as granted). It is understood, for this document, that the phrase “includes” is equivalent to the word “comprising.” The foregoing has outlined the non-limiting embodiments (examples). The description is made for particular non-limiting embodiments (examples). It is understood that the non-limiting embodiments are merely illustrative as examples.

What is claimed is:

1. A system for user skin color determination of a user, the system comprising:
a skin analysis assembly, configured to:
obtain a user skin image of the user, comprising one or more user skin image pixels;
perform color processing on the user skin image to arrive at a user skin color; and
output a result of the color processing, the result comprising the user skin color.

2. The system of claim 1 wherein the skin analysis assembly comprises a skin analysis device attached to a mobile device.

3. The system of claim 2 wherein the user skin image is at a magnification of not less than 10×.

4. The system of claim 1 wherein performing color processing further comprises:
determining an average pixel condition color score for the user skin image pixels in the user skin image; and
if the average pixel condition color score is below an image condition color threshold then:
calculating a per-pixel condition color threshold;
generating a condition color mask based on the per-pixel condition color threshold; and
removing pixels, from the user skin image pixels, that exceed the per-pixel condition color threshold; and
obtaining a user skin color from the user skin image pixels.

5. The system of claim 1 wherein performing color processing further comprises:
determining an average pixel redness score for the user skin image pixels in the user skin image; and
if the average pixel redness score is below a image redness threshold then:
calculating a per-pixel redness threshold;
generating a redness mask based on the per-pixel redness threshold; and
removing pixels, from the user skin image pixels, that exceed the per-pixel redness threshold; and
obtaining a user skin color from the user skin image pixels.

6. The system of claim 5 wherein the determining further comprises using a human perception mimicking algorithm developed using empirical A/B testing results.

7. The system of claim 5 wherein the image redness threshold is based on one or more of a processing power of the system, a desired speed and a desired accuracy of the calculated user skin color.

8. The system of claim 5 wherein the calculating the per-pixel redness threshold is by testing various per-pixel redness threshold values and choosing the per-pixel redness threshold value that minimizes the delta E from skin images that already had LAB values.

9. The system of claim 1 wherein the system is further configured to:
apply a color transposition to the user skin color, based on a transposition between the system and an alternative skin color system, and wherein the result further comprises a transposed user skin color.

10. The system of claim 9 wherein the system is further configured to:
provide a product recommendation, based on one or more of the user skin color and the transposed user skin color, and wherein the result further comprises a product recommendation.

11. The system of claim 10 wherein the system is further configured to:
receive an empirical feedback, based on one or more of the product recommendation, the user skin color and the transposed user skin color.

12. The system of claim 1 wherein the system is further configured to:
calibrate the skin analysis assembly, by taking one or more images of a calibration card, to obtain a calibration transposition that is applied to the user skin image.

13. A method for user skin color determination of a user, the method comprising:
obtaining, by a skin analysis assembly, a user skin image of the user, comprising one or more user skin image pixels;
performing color processing on the user skin image to arrive at a user skin color; and
outputting a result of the color processing, the result comprising the user skin color.

14. The method of claim 13 wherein the skin analysis assembly comprises a skin analysis device attached to a mobile device.

15. The method of claim 14 wherein the user skin image is at a magnification of not less than 10×.

16. The method of claim 13 wherein the color processing further comprises:
determining an average pixel condition color score for the user skin image pixels in the user skin image; and
if the average pixel condition color score is below an image condition color threshold then:
calculating a per-pixel condition color threshold;
generating a condition color mask based on the per-pixel condition color threshold; and
removing pixels, from the user skin image pixels, that exceed the per-pixel condition color threshold; and
obtaining a user skin color from the user skin image pixels.

17. The method of claim 13 wherein the color processing further comprises:
determining an average pixel redness score for the user skin image pixels in the user skin image; and
if the average pixel redness score is below a image redness threshold then:
calculating a per-pixel redness threshold;
generating a redness mask based on the per-pixel redness threshold; and
removing pixels, from the user skin image pixels, that exceed the per-pixel redness threshold; and
obtaining a user skin color from the user skin image pixels.

18. The method of claim 17 wherein the determining further comprises using a human perception mimicking algorithm developed using empirical A/B testing results.

19. The method of claim 17 wherein the image redness threshold is determined based on one or more of a processing power of the system, a desired speed and a desired accuracy of the calculated user skin color.

20. The method of claim 17 wherein the calculating the per-pixel redness threshold is by testing various per-pixel redness threshold values and choosing the per-pixel redness threshold value that minimizes the delta E from skin images that already had LAB values.

21. The method of claim 13, the method further comprising:
applying a color transposition to the user skin color, based on a transposition between the system and an alternative skin color system, and wherein the result further comprises a transposed user skin color.

22. The method of claim 21, the method further comprising:
providing a product recommendation, based on one or more of the user skin color and the transposed user skin color, and wherein the result further comprises a product recommendation.

23. The method of claim 22, the method further comprising:
receiving an empirical feedback, based on one or more of the product recommendation, the user skin color and the transposed user skin color.

24. The method of claim 13, the method further comprising:
calibrating the skin analysis assembly, by taking one or more images of a calibration card, to obtain a calibration transposition that is applied to the user skin image.

25. A system for user skin color determination of a user, the system comprising:
a skin analysis assembly, configured to:
obtain a user skin image of the user, comprising one or more user skin image pixels;
perform color processing on the user skin image to arrive at a user skin color, wherein the color processing further comprises:
determining an average pixel redness score for the user skin image pixels in the user skin image; and
if the average pixel redness score is below a image redness threshold then:
calculating a per-pixel redness threshold;
generating a redness mask based on the per-pixel redness threshold; and
removing pixels, from the user skin image pixels, that exceed the per-pixel redness threshold; and
calculating a user skin color from the user skin image pixels; and
output a result of the color processing, the result comprising the user skin color.

26. The system of claim 25 wherein the system is further configured to:
perform a color transposition to the user skin color, based on a transposition between the system and an alternative skin color system, and wherein the result further comprises a transposed user skin color.

27. The system of claim 26 wherein the system is further configured to:
perform a product recommendation, based on one or more of the user skin color and the transposed user skin color, and wherein the result further comprises a product recommendation.

28. A method for user skin color determination of a user, the method comprising:
obtaining a user skin image of the user, comprising one or more user skin image pixels;
performing color processing on the user skin image to determine a user skin color;
and outputting the result of the color processing, the result comprising the user skin color.

29. The method of claim 28 further comprising: determining an average pixel redness score user skin image pixels in the user skin image; and if the average pixel redness score is below a image redness threshold then: calculating a per-pixel redness threshold; generating a redness mask based on the per-pixel redness threshold; and removing pixels, from the user skin image pixels, that exceed the per-pixel redness threshold; and calculating a user skin color from the user skin image pixels.

30. The method of claim 29, further comprising:
performing a color transposition to the user skin color, based on a transposition between the system and an alternative skin color system, and
wherein the result further comprises a transposed user skin color.

31. The method of claim 30, further comprising:
handling a product recommendation, based on one or more of the user skin color and the transposed user skin color, and wherein the result further comprises a product recommendation.
